(19) United States
(12) Patent Application Publication
(10) Pub. No.: US 2023/0188931 A1
(43) Pub. Date: Jun. 15, 2023

(54) DEVICE, A SERVER AND A SYSTEM FOR DETECTING ITEMS OR PERSONS COMING INTO PROXIMITY OF ONE ANOTHER

(71) Applicant: VOXP PTE. LTD., Singapore (SG)

(72) Inventors: Dennis Beltran MENDIOLA, Ventura, CA (US); Jason Escano SIA, Manila (PH)

(73) Assignee: VOXP PTE. LTD., Singapore (SG)

(21) Appl. No.: 17/912,043

(22) PCT Filed: Aug. 12, 2020
(86) PCT No.: PCT/SG2020/050467

§ 371 Date: Sep. 16, 2022

(30) Foreign Application Priority Data
Mar. 17, 2020 (SG) 10202002470V

(51) Int. Cl. H04W 4/02; G16H 50/80; H04W 4/029

(52) U.S. Cl. CPC H04W 4/023; G16H 50/80; H04W 4/029

(57) ABSTRACT

A system for detecting items or persons coming into proximity of one another is disclosed. The system includes at least one scanning device and a server. Each scanning device is configured to detect any transmitting devices in its proximity and record an identifier of each transmitting device and a date/time the transmitting device is detected. The scanning device uploads the recorded information to the server. The server uses the recorded information to identify users of the scanning and transmitting devices that are at least possibly exposed to a virus when one of the users is determined to be an infected case.

### TECHNICAL FIELD

This invention relates to a device, a server and a system for detecting items or persons coming into proximity of one another. More particularly, this invention relates to a device, a server and a system for detecting people who have come into proximity with one another such that when one of them is determined to be an infected case of an infectious disease, others who have come into close proximity with the infected case may be traced.

### BACKGROUND

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Viruses, such as the one that causes the COVID-19 disease, can spread and kill. Interruption of virus transmission in a community is premised on the early detection and prompt isolation of new cases. During an outbreak with established person-to-person transmission, it is known that new cases are more likely to emerge among contacts of an infected person. For this reason, it is critical that all potential contacts of suspected, probable and confirmed cases are systemically identified and put under observation for as long a period as the maximum incubation period of the particular virus from the last day of contact, for example 14-21 days for case of the SARS-CoV-2 virus. Immediate evacuation of potentially infectious contacts with signs and symptoms of the disease to designated treatment centres or to the nearest healthcare facilities prevents high-risk exposure. Contact tracing to identify exposed people is therefore one of the most effective outbreak containment measures.

Contact tracing however poses serious challenges. It requires a lot of human effort. Contact tracing typically involves contact identification, contact listing and contact follow-up. Contact identification is an essential part of epidemiologic investigation for all cases meeting the standard/surveillance case definitions of the disease. These cases are classified as suspected, probable or confirmed. Contact identification therefore begins from a case of known infection. Identification of contacts is done by asking about the activities of the infected case and the activities and roles of the people around the infected case since onset of illness. Although some information can be obtained from the infected case, much of the information will typically come from the people around the infected case. In some instances, the infected case will have died or have already been admitted to an isolation facility with limited access. Contact tracing typically involves obtaining the following information:
(a) All persons who lived with the case in the same households since the time the case may have been infectious.
(b) All persons who visited the patient either at home or in the health facility since onset of disease.
(c) All places and persons visited by the patient since the time the case may have been infectious e.g. office, public transportation, relatives, friends, etc. All these places and persons should be considered, and contacts identified.
(d) All health facilities visited by the patient since onset of illness and all health workers who attended to the patient without appropriate infection prevention and control procedures.

The contacts may then be notified of their potential exposure, may be referred to for testing and/or monitored for signs and symptoms of the disease. In other words, the conventional means for tracking being applied currently is through interviews of the patient and friends of the patients, tracking a trail of breadcrumbs that branches out in many directions and many lost with the passage of time.

Therefore, it can be appreciated that contact tracing is thus a massive exercise that is time and labour intensive and likely not thorough. To contain the spread of the disease, speed of response and identification accuracy is critical to the overall success of contract tracing. Manual contact tracing may not be fast enough.

There is therefore a need for a system which addresses, at least in part, one or more of the forgoing problems.

### SUMMARY

According to an aspect of the present disclosure, there is provided a system that includes at least one scanning device. Each scanning device is configured to detect any transmitting devices in its proximity and record an identifier of each transmitting device and a date/time the transmitting device is detected.

In some embodiments of the system, the scanning device is configured to further record at least one of a location of the scanning device when the transmitting device is detected; an activity of a user of the scanning device when the transmitting device is detected; a signal strength of a signal transmitted by the transmitting device when it is detected by the scanning device; and a physiological data of the user of the scanning device when the transmitting device is detected.

In some embodiments of the system, the system further includes a server and wherein the scanning device is configured to upload recorded information to the server.

In some embodiments of the system, the scanning device uploads recorded information to the server via at least one intermediary device.

In some embodiments of the system, the scanning device is configured to further receive a health status of the user of the scanning device and/or health status of users of devices that are determined to be in the vicinity of the scanning device.

In some embodiments of the system, the server is configured to trace from the recorded information to identify users of the scanning and transmitting devices that are at least possibly exposed to a virus when one of the users is determined to be an infected case.

In some embodiments of the system, the server is configured to further determine a location of each user based on the recorded signal strengths of a signal transmitted by the transmitting device that is detected by at least two scanning devices and the location of the at least two scanning devices; determine a distance between each user and the infected case based on the location of each user; and identify users that are at least possibly exposed to the virus based on the distance between each user and the infected case.

In some embodiments of the system, the identifier of a transmitting device is a mobile number or an inherent address of the transmitting device.

In some embodiments of the system, the server includes a user contact information associated with each inherent address of the transmitting device.

In some embodiments of the system, the system further includes a registration device that is configured to detect signals from the transmitting device to read the inherent address thereof; receive from a user of the transmitting device the user contact information to be associated with the inherent address; and send the inherent address and associated user contact information to the server.

In some embodiments of the system, the scanning device is either installed in a fixed location or mobile.

According to another aspect of the present disclosure, there is provided a scanning device that is part of the above-described system.

According to another aspect of the present disclosure, there is provided a server that is part of the above-described system.

Other aspects and advantages of the invention will become apparent from the following detailed description, taken in conjunction with the accompanying drawings, illustrating by way of example the principles of the invention.

### BRIEF DESCRIPTION OF DRAWINGS

The invention will be better understood with reference to the drawings, in which:

FIG. 1 is a block diagram of a system including a scanning device and multiple transmitting devices according to an embodiment of the invention;

FIG. 2 is a block diagram of a system similar to the system in FIG. 1 with smartphones being used as scanning and transmitting devices;

FIG. 3 is chart showing typical symptoms of a COVID-19 infected person;

FIG. 4 is user interface screen of a smartphone in FIG. 2;

FIG. 5 is a block diagram of a system according to another embodiment of the invention; and

FIG. 6 is a block diagram of a system according to yet another embodiment of the invention.

### DETAILED DESCRIPTION OF THE EMBODIMENTS

Throughout this document, unless otherwise indicated to the contrary, the terms “comprising”, “consisting of”, “having” and the like, are to be construed as non-exhaustive, or in other words, as meaning “including, but not limited to.”

Furthermore, throughout the specification, unless the context requires otherwise, the word “include” or variations such as “includes” or “including” will be understood to imply the inclusion of a stated integer or group of integers but not the exclusion of any other integer or group of integers.

Unless defined otherwise, all technical and scientific terms used herein have the same meaning as is commonly understood by a skilled person to which the subject matter herein belongs.

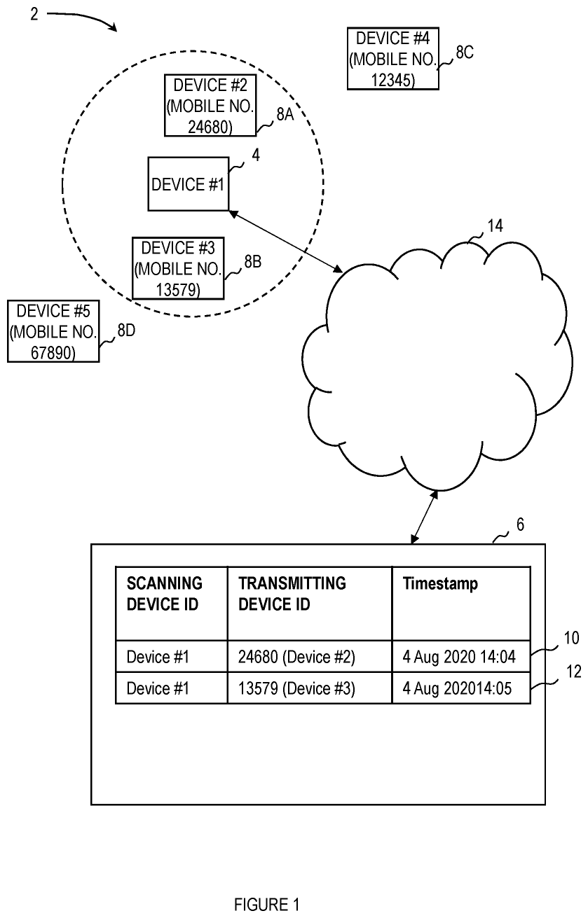

As shown in the drawings for purposes of illustration, the invention may be embodied in a novel, efficient and thorough contact tracing system. Existing systems tend to be labour intensive. Referring to FIG. 1, a scanning device embodying the invention is generally configured to detect any transmitting device in its proximity; and to record an identifier of each transmitting device in its proximity and a date/time the transmitting device is detected. A scanning device and a transmitting device may be one and the same device. A device may alternate between the role of a scanning device and a transmitting device. A server embodying the invention is generally configured to receive the recorded information from at least one scanning device. The server may be a physical server or a virtual server, such as a cloud server.

Specifically, FIG. 1 shows a system 2 for creating contact events and using the contact events for contact tracing and possibly other applications. The system 2 includes at least one scanning device 4 that is data communicatively coupled to a server 6. The scanning device 4 is able to detect any transmitting device 8A-8D that comes into a proximity of the scanning device 4. The scanning device 4 and the transmitting devices 8A-8D may be any device that is able to communicate via a wireless protocol. The scanning device 4 may be a laptop, tablet, smartphone, specifically designed device, etc. The wireless protocol may include, but not limited to, the Bluetooth, Bluetooth Low Energy (BLE), Wi-Fi and other suitable protocols. However, when it is required to detect devices that are in closer proximity, such as within a metre or two of each other, Bluetooth Low Energy (BLE) may be more appropriate. Bluetooth also has the advantage of low power consumption and users are therefore more likely to leave Bluetooth transmission on their transmitting devices 8A-8D always on.

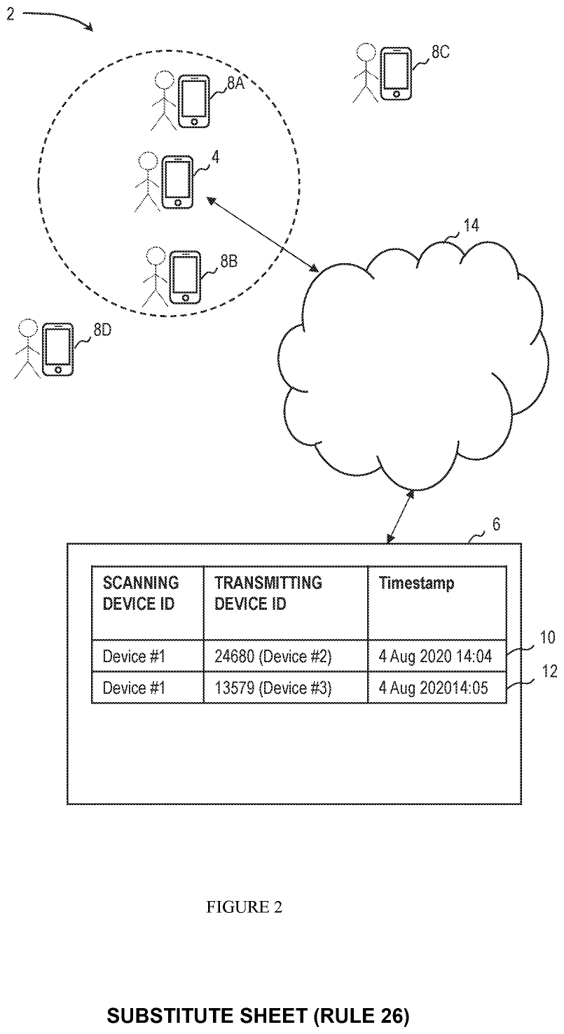

In this embodiment, the scanning device and the transmitting device may be smartphones having an App running thereon either in a foreground or background as shown in FIG. 2. Hereinafter, the scanning device 4 and the transmitting device 8A-8D will be referred to as a scanning smartphone 4 and a transmitting smartphone 8A-8D respectively. When the App is first run, a user will be requested to enter his contact details. An example of contact details may be the mobile number of the smartphone or another phone. However, other contact details may also be used, such as but not limited to, a home telephone number, an email address, a physical address, a post box address, etc. After the user enters his mobile number, the App generates an authentication code and sends the authentication code to the entered mobile number. The user will receive the authentication code on his phone and he can then enter the received authentication code into the App. In this manner, the App is able to verify that the mobile number is indeed a valid one and belongs to the user. Once such a verification step is completed, the App is able to operate the smartphone as a transmitting device 8A-8D. The mobile number also functions as an identifier (ID) of the smartphone 4, 8A-8D, or more specifically, the user of the smartphone 4, 8A-8D.

The App on the scanning smartphone 4 goes into a receiving mode where it monitors BLE channels for transmission of messages by transmitting smartphones 8A-8D. Each message will contain the mobile number associated with the transmitting smartphone 8A-8D. Once the scanning smartphone 4 detects that there are transmissions by any transmitting smartphones 8A-8D, the scanning smartphone 4 will receive the transmitted message to obtain the mobile number. Take for example the smartphones shown in FIG. 2. At an instant in time, Device #1 will be operating as a scanning smartphone 4 and it will be in a receiving mode and the other smartphones 8A-8D, Device #2-Device #5, operating as transmitting smartphones 8A-8D will be in a transmitting mode. The scanning smartphone 4 will be able to receive messages transmitted by the transmitting smartphones 8A, 8B, Device #2 and Device #3, that are within its range, i.e. within a proximity of the scanning smartphone 4. Devices #4 and Device #5 are outside the range of Device #1 and are therefore not detected by Device #1. As mentioned above, BLE typically has a short signal range of about 2 m. Therefore, the scanning smartphone 4 can only receive transmissions from transmitting smartphones that are within a 2 m radius or thereabouts of the scanning smartphone 4. When the scanning smartphone 4 receives a message from a transmitting smartphone 8A-8D, the scanning smartphone 4 records in its memory 1) the mobile number of the transmitting smartphone 8A-8D and 2) a timestamp that includes the day and time the instant the message was received by the scanning smartphone 4, i.e. when the transmitting smartphone 8A-8D was detected. In the example shown in FIG. 2, the scanning smartphone 4 will create two records, a first record 10 including a mobile number 13579 and a timestamp when transmitting smartphone 8A, Device #2, is detected; and a second record 12 including a mobile number 24680 and a timestamp when transmitting smartphone 8B, Device #3, is detected. This recorded information is useful for indicating that Device #2 and the Device #3 are in close proximity to Device #1 at the day and time indicated by the respective timestamps. The scanning smartphone 4 will monitor the BLE channels for transmissions by the transmitting smartphones 8A-8D for example at regular intervals of 1, 2, 5, 10, 15 seconds or longer as appropriate. Each time the scanning smartphone 4 detects that a transmitting smartphone 8A-8D is in its proximity, the scanning smartphone 4 will create a record as described above. Over time, many records may be created on the scanning smartphone 4 for the transmitting smartphones 8A-8D that came into close proximity at some point in time to the scanning smartphone 4. Based on these records on the scanning smartphone 4 alone, it is possible to know for how long each transmitting smartphone 8A-8D was within the proximity of the scanning smartphone 4. One possible application for maintaining such records is contact tracing to prevent the spread of a disease. For example, if the user of the scanning smartphone 4 is diagnosed to have contracted a disease and may have been infectious starting from a certain date, those people who have come into contact with the user, i.e. in close proximity to the confirmed case and therefore may have been exposed, can be easily identified using the records maintained on the scanning smartphone 4. The mobile numbers can be retrieved from the records created for a predetermined period when the user is determined to be likely to be infectious, so that the contacts may be identified and contacted for any necessary follow-up. In this manner, contacts having smartphones running the App will not be missed during contact tracing. While it is only described that records are created on the smartphone 4, Device #1, in FIG. 2, it should be noted that each of the other smartphones 8A-8D will also be creating records of smartphones that are in their proximities when they go into their respective receiving modes. That is, while Device #1 records that Device #2 is in its proximity, Device #2 also records that Device #1 is in its proximity.

While the information recorded in a scanning smartphone 4 may suffice for contact tracing, the information may be erased or the scanning smartphone 4 may be misplaced or stolen, in which case, contact events information will be lost. In one embodiment, the scanning smartphone 4 sends the recorded information via the Internet 14 to the server 6. Each record that is sent to the server 6 includes an identifier of the scanning smartphone 4, such as a mobile number of the scanning smartphone 4. Therefore, each record in the server 6 will include the following fields: 1) scanning smartphone identifier, 2) transmitting smartphone identifier, and 3) timestamp. The scanning smartphone 4 may send each record to the server 6 as and when the record is created at the scanning smartphone 4, i.e. in real time. Alternatively, the scanning smartphone 4 may send records to the server 6 at intervals. When the sending of records fail, for example when connection to the Internet 14 is lost, the scanning smartphone 4 will resend the records until it succeeds in sending the records to the server 6. In another embodiment of the uploading process, information collected by a scanning smartphone 4 without quality internet connection may be relayed via bluetooth, ad hoc Wi-fi or any other suitable protocol to another smartphone that has better internet connection. Or this could be a chain of one or more intermediary smartphones relaying information in hops to those smartphones that have good internet connection. Examples of methods that can be used for such relaying of information include, but is not limited to, Life Messenger and the method described in U.S. Patent Publication No. 20180220353, entitled “System and Method for Relaying Information”, to VOXP Pte Ltd.

The server 6 may be administered and operated by a national, regional, or district health authority. The health authority may access the records on the server 6 to carry out contact tracing when it is ascertained that a person is an infected case, for example, of the SARS-CoV-2 virus. COVID-19 is used as an example of an infectious disease in this disclosure. It should, however, be appreciated that contact tracing may be used for other types of infectious disease as well. It is known that COVID-19 spreads by symptomatic, pre-symptomatic and possibly asymptomatic transmissions. A symptomatic COVID-19 case is a case who has developed signs and symptoms compatible with COVID-19 virus infection. Symptomatic transmission refers to transmission from a person while they are experiencing symptoms. There has been evidence that COVID-19 is primarily transmitted from symptomatic people to others who are in close contact through respiratory droplets, by direct contact with infected persons, or by contact with contaminated objects and surfaces. Data has shown that shedding of the COVID-19 virus is highest in the upper respiratory tract (nose and throat) early in the course of the disease, i.e. within the first 3 days from onset of symptoms. Preliminary data suggests that people may be more contagious around the time of symptom onset as compared to later on in the disease.

The SARS-CoV-2 virus may also spread by pre-symptomatic transmission. The incubation period for COVID-19, which is the time between exposure to the virus (becoming infected) and symptom onset, is determined to be on average 5-6 days. However, it has been seen in some cases that it can be up to 14 days. During this period, also known as the “pre-symptomatic” period, some infected persons can be contagious. Therefore, transmission from a pre-symptomatic case can occur before symptom onset. Some data suggests that some people can test positive for COVID-19 from 1-3 days before they develop symptoms. Thus, it is possible that people infected with COVID-19 could transmit the virus before significant symptoms develop. Pre-symptomatic transmission still requires the virus to be spread via infectious droplets or through touching contaminated surfaces.

SARS-CoV-2 virus may further spread by asymptomatic transmission. An asymptomatic case is a person infected with COVID-19 who does not develop symptoms. Asymptomatic transmission refers to transmission of the virus from a person, who does not develop symptoms. Not much yet is known about asymptomatic transmission. This does not exclude the possibility that it may occur. Asymptomatic cases can therefore be reported as part of contact tracing efforts if required.

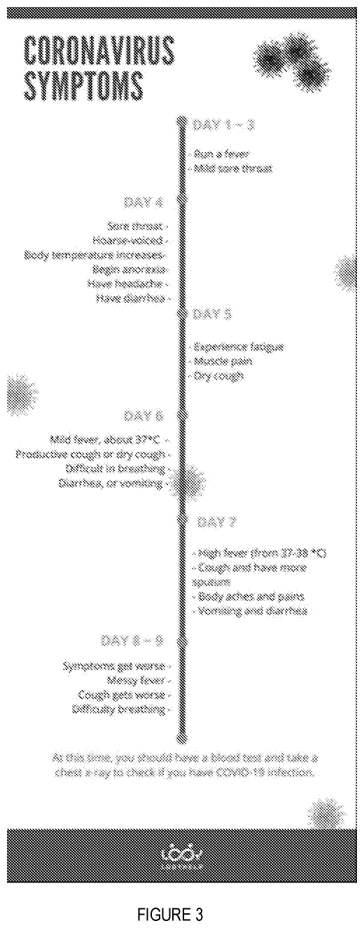

Contact tracing can be carried out using the data in the records stored in the server 6. For example, if a person is tested COVID-19 positive today, it is possible for the server 6 to determine from his existing symptoms approximately how many days have passed since the onset of symptoms based for example on the chart shown in FIG. 3. The server can then add a predetermined number of days, e.g. fourteen days, to the number of days since onset of symptoms to arrive at a total number of days for contact tracing. For example, if the person is determined to be having a high fever, he is probably about seven days since onset of symptoms. Adding fourteen days to that gives twenty-one days. The server 6 is then used by the health authority to query records relating to the person as far back as twenty-one days ago to determine who came into “close contact” with him during that period and therefore might have been exposed to the virus. These people can then be contacted, automatically via the server 6 or otherwise, for follow-up actions to be taken to curb the spread of the virus. As of the time of this disclosure, health authorities have begun to say that 10 days from the onset of symptoms, the infected person will not have enough viral load to infect others. If this becomes more widely accepted, the parameters for the contact tracing window maybe adjusted accordingly; in this particular case, to a much narrower window. In this manner, contact tracing can be automated and contact tracing can be carried out very quickly, effectively and thoroughly.

Although the incubation period is described above as being fourteen days long, it is possible over time, from more positive cases being detected and a determination of who and where they have contracted the virus from etc., for the server to determine via an artificial intelligence (Al) core running thereon and deep learning techniques that the incubation is shorter or longer than fourteen days. In that case, based on what is known about infections for the COVID-19 disease, the contact tracing period may be adjusted accordingly.

As described above, contact tracing may be for identifying persons who came into direct contact with the infected case, i.e. for only those having a first degree of separation from the infected case. However, contact tracing may also be extended to those having a second or higher degree of separation from the infected case, if necessary. For example, the server 6 may be configured to process up to two degrees of separation: Joe came into contact with Jane, and Jane came into contact with Mary. The server 6 will be able to identify Mary as a person who is potentially exposed to the virus that infected Joe. As with the case of the incubation period, the server 6 may be configured to contact trace to an appropriate degree of separation as deeper insight is gained of how the SARS-CoV-2 virus spreads.

And as described above, COVID-19 can be transmitted by close contact through respiratory droplets and contact with contaminated objects and surfaces. It is therefore also beneficial not just to know who came into close contact with each other, but also where they were and what they are doing so as to be able to more accurately identify suspected and probable cases. This additional information may be used by the Al core to target people at a higher risk of contracting COVID-19. For example, in a church where there is usually singing and in a restaurant where people sit close to each other and have prolonged conversations, there may be a higher risk of contracting COVID-19 as compared to people reading in a library or browsing in a shop. As another example, the risk may also be high in a gym where people work out moving from one exercise station to another. Hence, the context by which “contact” is made between two or more individuals is important in the assessment of how extensive the contact was. For example, in processing contact events by the Al core, greater weight may be placed on contact events/records between two people socializing at a bar and possibly sharing bar food compared to people simply walking past each other. In this regard, information such as the location of the scanning smartphone 4, and accelerometer and gyro sensor information captured by the scanning smartphone 4 when a transmitting smartphone 8A-8D is detected may further be recorded and used in contact tracing.

The type of establishment the scanning smartphone 4 is located may be determined based on the scanning smartphone's mapping functionalities, such as location-based services (LBS) data gathered from nearby cellular towers, its Wi-Fi connection, and GPS. Combining these location data enables a fairly accurate tagging of a user's location, even if he/she is indoors. Thus, the record may further include the location of the scanning smartphone 4 in addition to the ID of a transmitting smartphone 8A-8D and timestamp.

Alternatively, the record may include location information, such as GPS information, and the server may use Google Maps to determine from the location information the establishment where the scanning smartphone 6 was located, whether it was in a restaurant, a church, a gym, or in an outdoor setting.

Additionally or alternatively, the App may also record accelerometer and gyro sensor data collected by the scanning smartphone to better assess what the user is doing, e.g., if he is idle (having dinner, reading a book), walking around (socializing), or even sneezing or coughing (if a jerking motion is sensed). The accelerometer and gyro sensor information may also aid in determining the type of contact, e.g., one sharing the use of the same gym equipment. In such a case, in addition to the ID of transmitting smartphone and timestamp, the record may further include an activity that the user of the scanning smartphone 4 may be involved in when the transmitting smartphone 8A-8D is detected.

With the different information captured in a record, and with a sufficiently large number of people downloading and running the App, over time the number of records would grow to be statistically significant and contact tracing may be carried out more quickly and accurately as more knowledge is gained about the SARS-CoV-2 virus. The records may even be expanded to include other information so that contact tracing may further be enhanced by determining with a higher probability if a contact has contracted the virus. Such information includes, but is not limited to, strength of the signal received by the scanning device 4, which may be used to indicate the distance between the scanning device 4 and the transmitting device 8A-8D when it was detected by the scanning device 4. The data collected may even be used to discover ways by which the probability of a person contracting the virus may be decreased, and in the case of those already infected, lessen the severity of damage.

Some of these information captured may also be used, for example by a healthcare specialist, to determine the health status of a person, e.g. if a person is infected by the virus. For example, the scanning smartphone may receive information from wearables of the person that can be used further to determine if there are changes in the person's activity, mobility, jerking motions or sluggishness, heart rate, temperature, blood oxygen levels; information that could be used to determine if that person is possibly infected by the virus, maybe even earlier than when actual symptoms appear. In such a case, physiological data may be added to each record in the scanning smartphone for sending to the server 6. Alternatively, the physiological data may be separately sent to the server 6. The Al core in the server 6 can be constantly looking for patterns that would be more accurate in determining whether a person has in fact contracted the virus even earlier than the typical 11.5 to 14 day window. Further information, such as temperature and humidity if found to be relevant later, may be captured and added to each contact event where necessary.

An option in the App may further be provided for the user to fill up more data (optional at registration) so that better health assessments can be made. The user can, for example, also indicate if he is feeling certain things or showing symptoms, such as coughing or experiencing a shortness of breath. He can volunteer his medical history, etc. This data is kept private. Even if the user indicates that he/she strongly feels that he has been infected, unless the Red Cross, the W.H.O. and other certified attesters, confirm that indeed he/she is, the server 6 will not trigger contact tracing and/or a cascade of alerts to other users who had come into close contact with him.

Processing and analyzing the data gathered by scanning smartphones 4 may initially be performed on the cloud, using one or more powerful servers 6. As more knowledge is gained about the SARS-CoV-2 virus, it can be expected that there may come a time when accurate diagnosis can be made through edge computing, with limited if at all connection to the cloud.

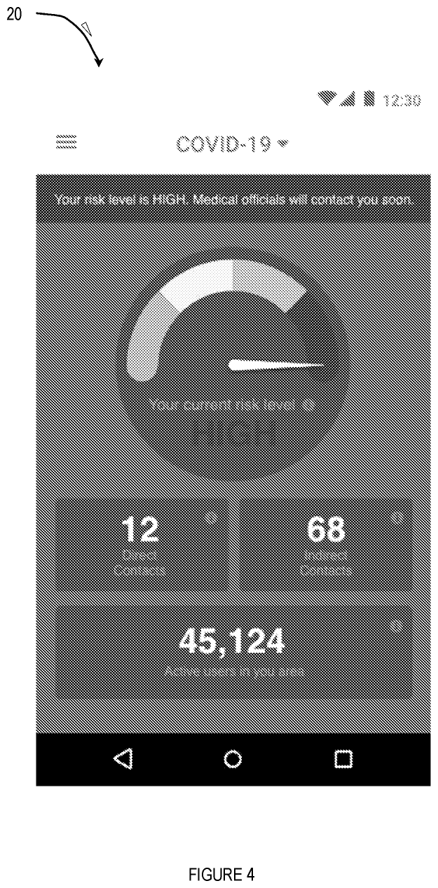

Additionally, the server 6 may be able to determine the likelihood/risk level of a person and/or his close contacts of having contracted the virus and to send this information to the scanning device 4 and the transmitting devices 8A-8D. The App will have a user interface (UI) screen 20 as shown in FIG. 4 for displaying the risk level/health status. The UI screen 20 includes a color risk meter, that goes from Green, to Yellow, and to stark Red as an example for showing the likelihood/risk level of the person contracting the virus. Green indicates that the person is likely healthy, i.e. at a low risk level of contracting the disease. Yellow indicates that there is a likelihood that the person may have contracted the virus, i.e. suspected or probable. The person may be advised to monitor his/her condition more closely, and to consult a healthcare specialist or professional if he/she is starting to feel unwell. Stark Red indicates to the person that he/she has been determined to be COVID-19 positive by a reputable agency, such as the Red Cross or W.H.O., i.e. a confirmed case. More colours, such as orange between the yellow and red sections, may be introduced if it is necessary to further distinguish the risk levels.

With this risk level and location information of each scanning smartphone 4, the server 6 may additionally or alternatively indicate to a person through his smartphone 4 if any infected persons or persons likely to be infected are in his vicinity. This may be done, for example, by indicating on a map including zones on the smartphone 4 showing where these people are. Names of these people will not be shown on the map. The exact locations of where these people are also not shown. This is to comply with data privacy rules. For example, the map may show concentric zones centred on where the smartphone 4 is with indicia thereon showing just the number of infected or possibly infected people in each zone. However, in an emergency situation, it is envisaged that exact locations where each infected or possibly infected person is may be shown on the map such that the user can try to avoid them.

Going back to contact tracing, when a person is flagged as of a RED health status/risk level, the server 6 does a back tracing by retrieving from the records those who came into close contact with the person. For example, in this embodiment shown in FIG. 2, the server 6 will retrieve all records corresponding to the mobile number (scanning device ID) of the infected person. From these records, the server 6 can then identify transmitting smartphones IDs (mobile numbers) as those who came into contact with the infected person. The server 6 may then send alerts/notifications to their mobile phones informing them that they may be at risk of contracting the virus, and hence, urging them to seek professional help, to have themselves checked, and to start monitoring their bodies more closely looking for symptoms. Their health status indicated on their devices may also be turned from Green to some gradation of Yellow (suspected or probable case). The deeper the Yellow is, the greater the likelihood that he or she has also been infected. And this may be determined by the context of the contact, i.e., where the contact occurred, what sort of activity the infected person was engaged in, how infectious the infected person was at the time of contact, how long the contact was, how far was he from the infected person, etc.

Optionally, the relevant authorities or government may encourage or require that people who are confirmed to be infected, i.e. whose risk meter points to RED, encourage people they have come into contact with to turn themselves Yellow. These people may indicate to the server 6 their new health status manually.

The health status may also be used for determining who is allowed to enter or leave an area under lockdown. When one has to enter or leave a lockdown area, all he has to do is show his App and if his risk meter shows a Green health status, he will be allowed through. There are several ways the integrity of the system can be protected, one of which is to tag the App to one's Facebook account. Since it is rather easy to tell if a Facebook account is real or not, a rule can be created such that the Facebook account to be tagged with the App must have been active for example for two years. Verification that this is one's smartphone is simply done by calling the mobile number of the smartphone from the Facebook app. In any case, the border patrol can deny entry at their discretion, if they sense something might be wrong. However, not all smartphones in developing countries have 24×7 internet connection. In this situation, the rules can be modified to accommodate a larger group of people entering or leaving a lockdown area. For example, in a car with five passengers including the driver, at least two must show an App with Green meter reading before the car is allowed through.

To assess the likelihood of one being infected, the server 6 may introduce weightages or scores. As described above, tracing may be extended beyond the direct contacts of the infected person. For example, User A may have an indirect contact with User J in 3 hops using a first trail of records. User A may also have an indirect contact to User J through 2 hops using a second trail of records. Both trails may be used to determine the likelihood of infection of User J. It is possible that one trail is given a higher weightage or score than the other trail. And within each trail, different weightages or scores may be given to contact events for User J depending on how substantial those contact events were.

Over time, more will be known about the SARS-CoV-2 virus; for example, how contagious it becomes over time or how the severity of a case is correlated with how contagious it is. Information gathered may be applied in deciding between which of two chains shall be used to determine User J's risk meter reading: the more infectious the case of User A in which contact with him was made by the next hop, the greater the weight that will be attached to this in determining User J's risk meter reading. At the time of this disclosure, health authorities suggest that a person is most infectious from the time of onset of symptoms to Day 3. If contact with User A occurred during this highly infectious state, the greater the weight that will be accorded this chain in determining User J's risk meter.

As more data is made available, the Al core in the server 6 may look into data of other users that resulted in more accurate assessments of the health condition of those users, and develop and apply rules for subsequent health assessments. At a later time, the Al core may then check if those rules applied gave more accurate assessments. The Al core repeats the process by applying another set of rules and comparing accuracy with those obtained using previously developed rules with the aim of increasing accuracy over time. Through Al, Deep Learning, and Big Data Analytics, the rules applied in these situations are updated and modified to improve the system's accuracy further.

Scoring can also be applied to certain users or segments of users that have been shown to deliver more accurate results. This may be the case if their smartphones are more sensitive and can collect more meaningful measurements due to the sophistication of its sensors, or the App users have allowed the App to access more data measured by the phone (e.g., allowing all-the-time access to location information versus “allow only when in use”), thereby being able to discern the “context” of the contact events. These user-smartphone tandems will be accorded a higher score.

Another application relates to using the contact events to track down patient zeros. From those who are confirmed infected, the records may be searched for common people these infected persons have come into contact with. These common people are then assessed, through human contact tracing, investigative questioning or otherwise, if they could likely be the source of infection, i.e. patient zeros. This information will be useful in analyzing how outbreaks or community transmission have taken place, providing health officials with valuable insights and guidance in dealing with the pandemic. The data gathered may then be used to identify possible patient zeros earlier so that they may be put up for testing, isolated and/or quarantined. Effectively identifying possible patient zeros can also lead to further identification and tracking of others who may be likely to be infected since their demographics may be similar and are likely in proximity with others. Age is definitely a big factor in determining whether people could be asymptomatic carriers, e.g. those in their late teens and twenties, for example.

As described above, the server 6 is configured to receive records from the scanning smartphones 4. The server 6 is able to create records corresponding to those captured by the scanning smartphones 4. Each record in the server 6 will include the ID of a scanning smartphone 4 from which records are received, date/time and transmitting smartphones 8A-8D in the proximity of the scanning smartphone 4. And as described above, the record may optionally include a location of the scanning smartphone 4, an activity of the user of the scanning smartphone 4, at least one physiological data of the user of the scanning smartphone, and strength of signal received from a transmitting device. Also as described above, smartphones may also act as relay points for transmitting information from smartphones without internet connection to those that have.

As data is subjected to change when more knowledge of the virus is available, the following parameters may be made configurable in the server 6:
1. Asymptomatic Transmission Period (the number of days), since the W.H.O. cannot pin down an exact number yet.
2. Stage of Infection when contact was made with another person.
3. Proximity to another person (via RSSI or signal strength of the Bluetooth connection) attached with the location that would suggest at what establishment the contact was made or under what circumstances.
4. Duration of proximity.
5. RSSI threshold for defining a contact event. Depending on known variables in a particulation location, the RSSI threshold and duration time calibrated. For example, in Mass transit systems, a lower RSSI threshold is set for defining a contact event. For a Jeepney where ventilation is good and no air conditioning, a higher RSSI threshold may be set. Conversely, if it is a private or private-hire transport with air conditioning, a lower RSSI threshold may be used. And in an emergency room where people are presumed to be wearing masks or PPE, and are extremely careful, a higher RSSI threshold may again be set.
6. The number of other people with the App running in a particular area. All things equal, the greater the density of users in a particular area, the more reliable the system's assessment becomes.
7. There may also be a section in which the user can simply put in writing or voice record what he is feeling at certain times.

Other variables and options that may be included in the server include, but is not limited to:
1. Proximity data between devices. The proximity data may provide a network view across time. This temporal spatial vector rendered as a network will provide a base visualization capability that may help bioinformatics analysts turn many scenarios on contact tracing and forecasting to project the potential transmission.
2. Location data may be used together with proximity data. Geological Information System data and other known COVID-19 positive cases may be used in identifying potential spread at the network level.
3. Time stamp—across all systems. The temporal spatial vectors may allow playback and “play forward” of potential spread scenarios. Scenario play may assist enterprises, NGOs and LGUs in planning their mitigation and response. Playback may be used to immediately identify tracks of patients recently infected by the virus.
4. Known infection information - known infection data from the D.O.H. (Department of Health), the W.H.O. will be used to ensure fidelity of the source to anchor the analyses.

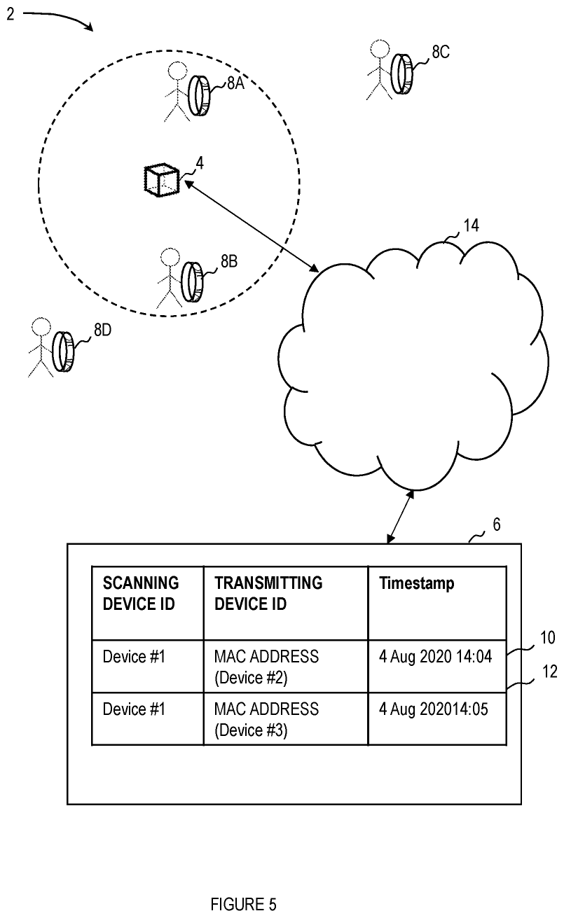

Although the embodiments described above are useful for contact tracing, they require each user to have a smartphone. In developing countries, not every person will be able to own a smartphone. It is therefore advantageous to use other devices as scanning and transmitting devices. In other embodiments, the scanning device may be any Bluetooth-based scanning device 4. For example, the scanning device 4 may be a purpose-built scanning device 4, as shown in FIG. 5, that is able to detect transmitting devices 8A-8D that comes into proximity of the scanning device 4. This scanning device 4 may for example be built out of small single-board computers. And the transmitting devices 8A-8D may be any non-mobile communication devices including, but not limited to, wearables, fitness devices, tracking devices, etc. Each of these devices 8A-8D has a unique device ID inherent to the device 8A-8D. For example, for a Bluetooth device, this unique device ID may be a Bluetooth address that is sometimes referred to as a Bluetooth MAC address. This Bluetooth address is a 48-bit value that uniquely identifies a Bluetooth device. Unlike the smartphones in the earlier described embodiments, these devices 8A-8D are not able to run the App described above. No mobile phone number information or other contact details can thus be included in the Bluetooth signals that are transmitted. These devices 8A-8D are devices that merely perform functions that they are designed for, receiving/transmitting beacons in the process, and are not modified to serve as tracker devices. A specific example of such devices is a Fitbit™ fitness tracker bracelet. It uses BLE to communicate with a smartphone and upload activity records. It also regularly broadcasts its presence so that its host smartphone can find it and connect with it. These broadcasts by the Fitbit™ bracelet may be detected by a scanning device 4 so that the scanning device 4 knows the Fitbit™ bracelet is within its proximity. The scanning device 4 may continuously monitor the Bluetooth channels for transmissions by these transmitting devices 8A-8D. In this manner, the transmitting devices 8A-8D that are within the coverage area of a scanning device 4 at about the same time may be deemed to be in close contact with each other and as such constitute contact events.

For the purpose of identifying the users of these transmitting devices 8A-8D, it is necessary to associate each transmitting device 8A-8D with a contact detail, e.g. mobile number, of the user of the transmitting device 8A-8D in a registration process. Other contact details may be used such as, but not limited to, mobile identification number (MIN) and a number of a device capable of receiving notification, for example, in the form of text messages. This registration may for example be carried out using a registration device, e.g. a smartphone. The smartphone may be the user's smartphone or someone else's smartphone. Alternatively, the registration device may be a device that runs web apps, e.g. the typical modern website browser like the Opera. The smartphone is brought in close proximity to the Fitbit™ bracelet. A Linking App running thereon is put on discover-nearby-devices mode, wherein the registration device broadcasts a signal that is targeted at a Fitbit™ bracelet for registration. Upon receiving the signal from the registration device, the Fitbit™ bracelet for registration will respond by transmitting BLE signals. Other devices that may interfere with these BLE signals or that may appear as another BLE transmitting device are put away or turned off so that the Fitbit™ bracelet for registration may be clearly and unambiguously identified. The App can therefore see the nearby Fitbit™ bracelet, and since it is beside the smartphone, it would appear as the only device or one with the highest signal strength if there are other devices around. If a BLE device transmits its model information, the App may also display the model information to aid in device identification and selection. For example, a Fitbit™ bracelet may indicate its model name, such as “Inspire HR”. The Fitbit™ bracelet to be registered is selected on the registration device. The wearer of the Fitbit™ bracelet may then enter his particulars and credentials into the App, including his textable mobile number. The particulars entered may include a photograph of the user. Optionally, a one-time PIN (OTP) may be sent to the mobile number. The user can then enter this OTP into the App. This completes the registration and the App may show a status of “LINKED” and the user may receive a text message confirming the successful linking of his Fitbit™ bracelet to his mobile number. The information is sent to the server 6 so that the user of the Fitbit™ bracelet is traceable based on its Bluetooth address. Optionally, a device name of the BLE device is also captured if it is transmitted. The Linking App may also be a feature of the contact tracing App described earlier.

Instead of obtaining the Bluetooth address through transmissions from a device, the Bluetooth address may be encoded in a QR code that is attached to, printed on, or stamped on a BLE device. Linking may be carried out simply by reading the QR code; signal interference from other BLE devices will no longer be a concern and registration is likely to take a shorter time. With the Bluetooth address stored as an identifier of a user, the QR code of his BLE device may be read and used as a key to obtain the user's record, for example, to pull out his photograph for identification purposes and to determine his health status so as to know if he is to be held in quarantine for further investigation, for example.

During use, one or more scanning devices 4 may be placed at a location for continuously scanning for nearby transmitting devices 8A-8D that have come into their proximity and logging data associated therewith. The data may be used to discern how many of these transmitting devices 8A-8D are within the vicinity of each other right around the same time, for how long and approximately how densely or loosely packed they were. This information may be used for contact tracing as described above even in the absence of apps running on smartphones. With only the Bluetooth addresses captured, such a solution also addresses data privacy concerns given its anonymity.

For illustrative purposes, as shown in FIG. 5, a scanning device 4 may be a single board computer (SBC), such as a Raspberry Pi, with BLE features and Wi-fi that allows it to access a central server 6 via the Internet or WAN. The scanning device 4 may also be smartphones running iOS or Android operating systems, tablets (iPads and Android-based), laptops (with built-in Bluetooth, Wi-Fi, and/or LTE/3G), most single board computers marketed commercially, such as the Raspberry Pi, or the dongle compute sticks (Intel stick), which are ubiquitous and more likely adopted for use.

As for the transmitting devices 8A-8D, they may be any device that can transmit/publish/advertise Bluetooth signals containing information about what it is, who it is (e.g. Bluetooth addresses), and what it is trying to do (“find me”). These devices include, but is not limited to, purpose built tracker devices, fitness trackers such as Fitbit™ bracelets, smart watches, smart ID tags (those that come with BLE and NFC wireless features), smart medical tags for patients and the elderly, tracker/finder devices such as the Tile™ family of trackers, and legacy 2G/3G phones that come with Bluetooth. Essentially, any portable device that is typically handled only by one person and capable of transmitting BLE signals may be used as a tracked device.

As described above, it is important to know the context in a contact event between two people. One important information is where the contact took place. For example, if contact took place in an indoor situation, such as in a restaurant or nightclub, the probability that viral transmission occurred is likely to be higher than outdoor contact. Therefore, the location where the scanning device is installed may be sent to the server 6 as well.

In one embodiment, the scanning device 4 may be a single board computer, e.g. a Raspberry Pi, equipped with BLE and Wi-Fi. The scanning device 4 is placed at the main entrance of an establishment, e.g. a restaurant, to continuously scan for any device 8A-8D emitting Bluetooth signals (BLE 4.0 and higher). As customers wearing their Fitbit™ devices 8A-8D enter the restaurant, BLE data transmitted by the Fitbit™ devices 8A-8D is captured and recorded by the scanning device 4, logging its Bluetooth address (MAC address) and time, and optionally the device type and RSSI (received signal strength indicator). With this information captured by the scanning device 4, it is possible to know at a particular time who was at the restaurant. It would also be possible to determine how long they have been in the restaurant together. The RSSI may be indicative of how close the Fitbit™ device 8A-8D is to the scanning device 4. For example, a RSSI reading of −20 dBm captured by the scanning device 4 may be indicative that the Fitbit™ device that is transmitting the signal is about 3-6 feet (one to two metres) from the scanning device 4. With such additional information, it may be possible to determine with a higher accuracy the distance between two Fitbit™ devices that are in the proximity of the scanning device 4. This is especially so if the type of each Fitbit™ device is known and RSSI for the Fitbit™ device is calibrated.

If the restaurant has a large floor area for accommodating a large seating capacity, multiple scanning devices 4 may be installed at strategic locations in the restaurant. By having more than one scanning device 4, it is possible to tell more accurately the proximity of one person to another. This is done by approximation of how close each person is to the scanning devices 4 based on the RSSI of signals received from that person's transmitting device 8A-8D. For example, if signals from two FitbitT™ devices produce the same RSSI readings in different scanning devices 4, there is a likelihood that the two devices are located close to each other. As another example, the distance between two devices may be obtained by first determining the exact location of each device using the RSSI readings. Also, in more sophisticated implementations, “trilateration” based on RSSI could be used to more accurately determine the person's location within the restaurant. As such, when the exact location of people is known, proximity, i.e. distance between them can be better assessed. Trilateration involves at least three scanning devices 4. But even with just two scanning devices 4, more accuracy can still be achieved compared to having just one single scanning device 4. With two scanning devices 4, a person can be at any one of only two points of the intersection of radio pathways of the two scanning devices 4. With more accurate determination of one's location within the restaurant, it is possible to also tell if social distancing guidelines are being followed, e.g., maximum number of people allowed per section in the restaurant.

However, trilateration using RSSI is prone to errors. RSSI readings are different depending on the type of transmitting device 8A-8D and scanning device 4. There are also many factors that determine read signal strengths. In other words, there is no fixed correlation between signal strength and distance between a scanning device 4 and a transmitting device 8A-8D. For example, turning one's back to a scanning device 4 can dramatically change the scanning device's RSSI reading of signals from a transmitting device 8A-8D.

There are several alternative approaches to improving accuracy. One such approach is by measuring the distance of the transmitting device 8A-8D to the scanning devices 4 by measuring the time a particular signal is received by the different scanning devices 4. The intersection of the circular distances from the scanning devices 4 determine the exact location of the person. This approach requires that the internal “clocks” of the scanning devices are synchronized to a high level of precision, such as by anchoring it to the same GPS clock.

Contact events are thus defined by which transmitting devices 8A-8D were read by the scanning devices 4, who was wearing the transmitting devices 8A-8D, how long they were together, and where the scanning devices 4 are located in the restaurant. And as described above, in cases where other sensor data of a transmitting device 8A-8D, such as a fitness tracker, is exposed to the scanning device 4, it may be possible to further tell what kind of interaction is taking place, for example, people are sitting still having dinner, or standing up and socializing, dancing, etc. These kinds of information would be helpful in assessing the significance of a contact event in possibly triggering a viral transmission. And as mentioned above, if information such as temperature and humidity is found to be relevant, sensors may be added to the scanning device 4 to capture such information and add them to a contact event. As a further example, sound at the contact event may also be captured if proven useful.

From time to time or even in real time, the scanning devices 4 will upload information regarding the contact events to the server 6. The server 6 will, with the aid of Al and big data techniques, analyze the information so as to determine which contact events warrant further investigation to determine who is likely to be infected with the virus when a person in the restaurant they have come into contact with is confirmed to be COVID-19 infected. Alerts will be sent to those that are determined likely to be infected using text messages instead of via notifications and risk meter readings.

The scanning device 4 may use Wi-Fi to connect to the server 6. In other embodiments, a scanning device 4 may use IPWAN wireless technology, such as LoRaWAN and NB-IOT, which is more flexible for deploying in areas where internet connection is not readily accessible and Wi-Fi cannot reach those distances.

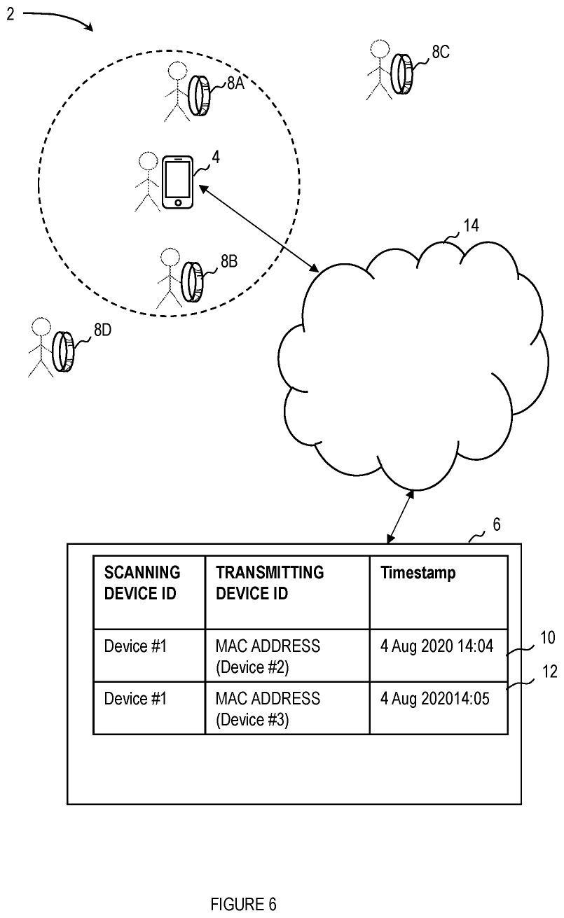

In another embodiment shown in FIG. 6, the scanning device 4 may be mobile instead of being installed at fixed locations as described above. In such an embodiment, the scanning device 4 may be a smartphone (iOS or Android based) with a scanning feature running thereon. This scanning feature may be integrated into existing contact tracing apps, such as that described above, that may already enjoy a substantial installed base of users. The smartphone, like the single board computer scanning device 4 described above, will continuously scan for BLE signal transmitting devices in its proximity.

As an example, the smartphone 4 as a scanning device 4 may detect a person with a first registered/provisioned BLE transmitting fitness bracelet 8A-8D come near it. The smartphone 4 may subsequently detect another person with a second fitness bracelet 8A-8D coming near it at about the same time. In this situation, the three people—the one with the smartphone 4 and the two with the fitness bracelets 8A-8D are determined to be close to one another and in a protracted contact with each other, for example, 10-15 minutes at about 2 meters apart. This information will be recorded as a contact event as described above, including who the three people are and how long they were together using timestamps. And optionally, the information may include where the contact took place, to the extent that the transmitting devices 8A-8D enable access by the smartphone App to the other sensory capabilities of the transmitting devices 8A-8D, what activity they might have been engaged in during the contact event, RSSI readings as a proxy for proximity and/or the type of smartphone and tracked devices. An example of other information that may be uploaded by the transmitting devices 8A-8D include physiological data, if so made available by the manufacturer or developer of the devices.

Location information where contact took place may be obtained using GPS and location based services (LBS) features of the smartphone 4. This is how a geographical context can be added to the contact event. Another advantage of using a smartphone 4 as a scanning device is that it has access to a uniform “clock” that may be the same one being used by other smartphone scanning devices 4 nearby. Trilateration geo-pinning of a tracked person using this uniform “clock” can be more easily achieved using the relative times a radio signal from a transmitting device 8A-8D is heard by the different smartphone scanning devices 4.

For trilateration to work, the smartphones 4 nearby must be on the same mobile network using the same precise “clock” and the location of the smartphone scanning specified by GPS rather than LBS. LBS is not so suitable at pinning people to a location, with margins of error in the tens of meters and even hundreds of meters depending on the density of nearby GSM mobile telecommunication towers. The contact event data is uploaded via the internet to the server 6 for further contact tracing and other purposes as described above.

Advantageously, the above described embodiments allow quick, systematic, efficient and thorough contact tracing. And with location and other data, there is also the possibility of being able to more accurately determine if a person is likely to contract a disease from an infected case the person came into contact with.

Although the present invention is described as implemented in the above described embodiments, it is not to be construed to be limited as such. For example, in one of the embodiments, it is described that the scanning device 4 send contact records to the server as and when records are created and each record includes the ID of a transmitting device 8A-8D and a timestamp. The scanning device 4 may however, send only a record when a transmitting device 8A-8D first comes into the proximity of the scanning device 4 and then sends another record when the transmitting device 8A-8D is determined to have left the proximity of the scanning device 4. From these two records, the server 4 will be able to determine the period the transmitting device 8A-8D is in the proximity of the scanning device 4. In this manner, the scanning device 4 need not send a record every few seconds to the server for the same transmitting device 8A-8D that remains in its proximity. In another embodiment, the scanning device 4 may simply track the start and end time that each transmitting device 8A-8D remains in the proximity of the scanning device 4, further reducing the number of records stored in the server 6.

As another example, it is described in one of the embodiments that a user is only required to enter his mobile number to be able to use the App. However, this should not be construed to be so limited. It is envisaged that more personal data may be required to fine tune and accelerate the Al core's learning process.

While a system specific to contact tracing in the current COVID-19 pandemic is described, the same system, devices and configuration of devices may be deployed in all future occurrences of epidemic. The system and devices may also be used in other applications such as, but not limited to, “finding missing devices”, “permission-based” tracking of marketing metrics, location-based advertising, detective work, criminology and security services, etc.

It should be further appreciated by the person skilled in the art that one or more of the above modifications or improvements, not being mutually exclusive, may be further combined to form yet further embodiments of the present invention.

1. A system comprising:
at least one scanning device, each scanning device being configured to:
detect any transmitting devices in its proximity; and
record an identifier of each transmitting device and a date/time the transmitting device is detected.

2. The system according to claim 1, wherein the scanning device is configured to further record at least one of:
a location of the scanning device when the transmitting device is detected;
an activity of a user of the scanning device when the transmitting device is detected;
a signal strength of a signal transmitted by the transmitting device when it is detected by the scanning device; and
a physiological data of the user of the scanning device when the transmitting device is detected.

3. The system according to claim 1, further comprising a server, and wherein the scanning device is configured to upload recorded information to the server.

4. The system according to claim 3, wherein the scanning device uploads recorded information to the server via at least one intermediary device.

5. The system according to claim 1, wherein the scanning device is configured to further receive at least one of:
a health status of the user of the scanning device; and
health status of users of devices that are determined to be in the vicinity of the scanning device.

6. The system according to claim 1, wherein the server is configured to use the recorded information to identify users of the scanning and transmitting devices that are at least possibly exposed to a virus when one of the users is determined to be an infected case.

7. The system according to claim 6, wherein the server is configured to further:
determine a location of each user based on the recorded signal strengths of a signal transmitted by the transmitting device that is detected by at least two scanning devices and the location of the at least two scanning devices;
determine a distance between each user and the infected case based on the location of each user; and
identify users that are at least possibly exposed to the virus based on the distance between each user and the infected case.

8. The system according to claim 1, wherein the identifier of a transmitting device is one of a mobile number and an inherent address of the transmitting device.

9. The system according to claim 8, wherein the server includes a user contact information associated with each inherent address of the transmitting device.

10. The system according to claim 9, further comprising a registration device that is configured to:
detect signals from the transmitting device to read the inherent address thereof;
receive from a user of the transmitting device the user contact information to be associated with the inherent address; and
send the inherent address and associated user contact information to the server.

11. The system according to claim 1, wherein the scanning device is one of installed in a fixed location and mobile.

12. A scanning device configured to:
detect any transmitting devices in its proximity; and
record an identifier of each transmitting device and a date/time the transmitting device is detected.

13. The scanning device according to claim 12, configured to further record at least one of:
a location of the scanning device when the transmitting device is detected;
an activity of a user of the scanning device when the transmitting device is detected;
a signal strength of a signal transmitted by the transmitting device when it is detected by the scanning device; and
a physiological data of the user of the scanning device when the transmitting device is detected.

14. The scanning device according to claim 12, configured to upload recorded information to a server.

15. The scanning device according to claim 14, wherein the scanning device uploads recorded information to the server via at least one intermediary device.

16. The scanning device according to claim 12, configured to further receive at least one of:
a health status of the user of the scanning device; and
health status of users of devices that are determined to be in the vicinity of the scanning device.

17. (canceled)

18. (canceled)

19. A server configured to receive from each of at least one scanning device:
an identifier of each transmitting device that is detected to be in a proximity of the scanning device; and
a date/time the transmitting device is detected.

20. The server according to claim 19, configured to further receive from the scanning device at least one of:
a location of the scanning device when the transmitting device is detected;
an activity of a user of the scanning device when the transmitting device is detected;
a signal strength of a signal transmitted by the transmitting device when it is detected by the scanning device; and
a physiological data of the user of the scanning device when the transmitting device is detected.

21. The server according to claim 19, wherein the server is configured to use the received information to identify users of the scanning and transmitting devices that are at least possibly exposed to a virus when one of the users is determined to be an infected case.

22. The server according to claim 21, wherein the server is configured to further:
determine a location of each user based on the received signal strengths of a signal transmitted by the transmitting device that is detected by at least two scanning devices and the location of the at least two scanning devices, and
determine a distance between each user and the infected case based on the location of each user;
identify users that are at least possibly exposed to the virus based on the distance between each user and the infected case.

23. (canceled)

24. (canceled)
